(19) United States
(12) Patent Application Publication
(10) Pub. No.: US 2011/0270282 A1
(43) Pub. Date: Nov. 3, 2011

(54) SURGICAL SUTURE POSITIONING SYSTEM FOR CLOSING AN OPENING INSIDE A TISSUE WALL

(76) Inventor: Norbert Lemke, Puchheim (DE)

(21) Appl. No.: 13/126,884

(22) PCT Filed: Oct. 29, 2009
(86) PCT No.: PCT/DE2009/001532

§ 371 Date: Jul. 7, 2011

(30) Foreign Application Priority Data
Oct. 29, 2008 (DE) 10 2008 053 809.4

(51) Int. Cl. A61B 17/04

(52) U.S. Cl. 606/148

(57) ABSTRACT

Described is a surgical suture positioning system for closing an opening inside an intracorporeal tissue wall, comprising an elongated shaft, which has a distal shaft region, on which a means is provided, which is made at least partially of an elastomer material and which can be reversibly transferred from a position that is flush with the shaft into a position radially protruding over the shaft, and a needle guide unit, which is provided on the shaft and in which at least one needle arranged movably along the shaft is guided, which needle can be reversibly transferred from a retracted position into an advanced distal position, in which the needle penetrates the means located in the position radially protruding over the shaft, wherein a suture carried along by the means remains in the means by retracting the needle into the retracted position. The invention is characterized in that the means has limbs that can be radially expanded away from the shaft and are operatively connected to an element such that the limbs can be reversibly transferred from the position that is flush with the shaft into the position radially protruding over the shaft.

### BACKGROUND OF THE INVENTION

1. Field of the Invention

The invention relates to a surgical suture positioning system for closing an opening inside an intracorporeal tissue wall, comprising an elongated shaft, which has a distal shaft region, on which a means is provided, which can be reversibly transferred from a position that is flush with the shaft into a position radially protruding over the shaft, and a needle guide unit, which at least in sections encloses the shaft radially and/or is integrated into the shaft, in which at least one needle, arranged movably along the shaft is guided, which needle can be reversibly transferred from a retracted position into a distal position, where it is operatively connected to the means located in the position radially protruding over the shaft.

2. Description of the Prior Art

Surgical instruments of the above type are used in cases, where closing of openings in tissue walls formed by incisions or cutting, for example in blood-carrying hollow vessels, has to be undertaken as quickly and patient-friendly as possible. Using such suturing apparatuses, the formation of hematomas can be effectively countered, especially since the use of compression or pressure bandages commonly used for the purpose of hemostasis at an independently healing vascular wall opening are no longer required to a large extent.

Suturing apparatuses of the type initially stated are known in multiple embodiments, and at the distal end of a shaft-shaped hollow instrument, which is suited for performing a locally minimal-invasive intervention, mostly have differently designed suturing needle applicators, to which it is possible to fasten one or several surgical sutures in the tissue area directly around the opening to be closed in a sling- or loop-type manner in order to ultimately close the opening mechanically by gently tightening the suture loop in order to support the natural healing process in an accelerating manner.

The suturing apparatus described in DE 693 34 017 T2 provides two suturing needles at the distal end of a hollow instrument, which can be displaced via the distal end of the hollow instrument and which can be radially expanded away from the hollow instrument, the needle ends of which are respectively connected with a continuous suture. The intracorporeal application of the suturing needles is undertaken such that the hollow instrument is passed through the tissue wall opening to be closed, wherein via a guide unit guided inside the hollow instrument the suturing needle apparatus is pushed out of the hollow instrument on the distal side and the suturing needles take a radially expanded position. Subsequently, the hollow instrument is retracted through the vascular wall opening to be closed, whereby a holding device is formed outside the vessel to be treated radially to the hollow instrument in an expanding, mesh-type fashion, into which the needle tips, once the needles have completely penetrated the tissue wall edge area surrounding the opening, engage in a barb-type fashion during the process of the needle arrangement being retracted. By complete proximal-side retracting of the hollow instrument, including the needle apparatus reinserted into its interior, the suture loop forming inside the vessel tightens and completely closes the vascular wall opening.

A further apparatus for closing a wound is described in DE 696 37 177 T2, which within a cannula provides a suture manipulator movable in the longitudinal direction, which is passed through a vascular wall opening to be closed. The suture manipulator provides expanding elements, through which a suture carried along is transferred into a radially expanded position along the cannula axis and can be caught by suturing needles designed in a barb-like shape, which penetrate the vascular wall from the outside in a vascular wall area surrounding the vascular wall opening. By proximally retracting the suturing needles as well as the suture manipulator, a suture loop closing the vascular wall opening is formed, which just like in the example described above mechanically closes the vascular wall opening by proximally directed pulling of the suture.

A further surgical suturing apparatus based on the principle explained above of “capturing” or “catching”, respectively, a suture loop inserted inside the vessel to be closed using a suturing needle designed in a barb-like shape is the subject of DE 698 35 444 T2. The known apparatus uses at least two needles, which are respectively forced through the tissue edge area around an opening to be closed. One of the two needles carries a suture guided in a loop-type manner, the other needle is pushed through the vascular wall without a suture and has a barb-shaped recess at its distal tip, with which the suture loop located inside the vessel is to be caught. Once the suture loop has been caught, both suturing needles are gently retracted, whereby a suture loop crossing the opening twice is formed, which in the same manner as with the apparatuses described above results in closing of the vascular opening by gentle tightening.

The apparatuses based on the principle described above of capturing or catching, respectively, an intracorporeally introduced suture loop are likewise described in DE 697 37 897 T2 as well as DE 699 25 185 T2.

A further known suturing apparatus for closing an intracorporeal vascular wall opening is described in DE 693 33 383 T2, which provides an endoscopic hollow instrument, at the distal instrument end of which two semi-circularly shaped needle holders are provided, which can be swiveled from a tight-fitting position to the instrument axis into a position radially expanded from the instrument axis. Through the needle holders, semi-circularly shaped needles are led, which at the needle tips the ends of a continuous suture deposited inside the distal endoscope tip are attached. By advancing the semi-circularly shaped needle tips, the inside of the vascular wall is penetrated by the tips and finally end up in a suture catching unit along the endoscope shaft. Once the needles as well as the needle holders have been returned into an initial position flush with the endoscope shaft, the endoscope-like suturing system can be extracorporeally removed, whereby a suture loop closing the vascular wall opening is formed.

A further generic apparatus improving handling and facilitating the execution of closing a tissue opening is the subject of DE 199 42 951 C1. In order to form a suture loop closing the tissue opening, the apparatus provides at its distal end area a suture guide unit, which in its longitudinal direction has a rear suture supply part, a front suture receiving part and an intermediate central suture release/suture clamping part. The central suture release/suture clamping part is, relative to the front suture receiving part, supported in a twistable fashion and furthermore has a cross-section that the suture release/suture clamping part enables in at least one rotating position the insertion of the sutures supplied from the rear suture supply part into the exposed receiving openings of the front suture receiving part. In a rotating position differing from the one stated, it is possible to hold the sutures received in the respective receiving openings together with the needles tight for pulling the entire suture guide unit out of the stated tissue wall opening.

With the fixedly specified spatial allocation of the three parts of the suture guide unit described above, the known apparatus guarantees clear and secure handling of the sutures for positioning a suture sling in the area of the vessel wall surrounding the opening.

U.S. Pat. No. 5,954,532 disclosed a surgical suturing apparatus in which the setup and function is illustrated in different embodiment variants in FIGS. 11 to 25. For producing a surgical suture sling in the area of a tissue opening to be closed, the surgical suturing apparatus provides a medical hollow instrument, inside which an interior shaft movable in the longitudinal direction is guided, the distal end of which protrudes over the hollow instrument on the distal side. At the area of the interior shaft protruding over the hollow instrument on the distal side, an elastomer element is attached, which can be transferred from an elongate position largely flush with the shaft into a shape radially protruding over the diameter of the hollow instrument by way of material compression or squashing, respectively, which can be initiated by retracting the shaft relative to the hollow instrument. In this shape of the elastomer element, two needles guided along the hollow instrument are deflected on the distal side, which penetrate the elastomer element expanded laterally to the hollow channel and in this manner simultaneously respectively insert a suture carried along into the interior of the elastomer, which retains the suture within the elastomer material due to the material-dependent frictional and restoring forces despite proximal retracting of the needle. In a next step, with distal advancement of the internal shaft, the elastomer material, which has expanded laterally to the hollow channel, is reshaped to be flush with the shaft.

In the application of the known surgical suturing apparatus, the elastomer element flush with the distal shaft area is passed through a tissue opening to be closed, wherein the surgical hollow instrument is positioned on the outside of the tissue opening with the needles attached thereto. With the radial expansion of the elastomer element and the subsequent needle advancement, the needles penetrate the tissue wall directly surrounding the tissue opening from the outside and subsequently enter the radially expanded elastomer element. With subsequent retracting of the needles, however, the suture respectively remains in the radially expanded elastomer element and thus also respectively in the penetration channels through the tissue wall caused by the needles. Finally, the suture ends, since they remain in the elastomer element, are transported in the proximal direction to the tissue opening through the tissue opening to be closed together with the elastomer element with the removal of the shaft, and in this manner forming a suture sling closing the tissue opening.

Compared to the surgical suturing apparatuses depicted above, the apparatus described in U.S. Pat. No. 5,954,732 represents a much more simplified form of a surgical suturing apparatus.

### SUMMARY OF THE INVENTION

Based on the state of the art according to U.S. Pat. No. 5,954,732 described above, the invention is a further improvement of user friendliness with a simultaneous guarantee of secure handling of any device components required for a suture sling closing the tissue wall opening. In particular, a generic apparatus is to be provided, with which it is possible to close tissue openings with large opening diameters, that is of more than 5 mm, as they occur with abdominal surgeries or similar surgical measures. Such big tissue openings cannot or not reliably be closed with the known surgical suturing apparatus, especially since the radial projection of the elastomer element achievable with material compression across the size of the tissue opening to be closed is too small. Since the tensile forces of the suture required for closing a tissue opening increase with an increasing size of the opening, a sufficiently large distance between the needle penetration channels through the tissue close to the edge and the tissue opening is required in order to reduce the cutting effect of the suture under tension on respective tissue areas.

The solution for the object the invention is based on is stated in claim 1. Characteristics advantageously further developing the invention are the subject of the dependent claims and may also be learned from the further description, in particular referring to the embodiments.

According to the solution, a surgical suture positioning system for closing an opening inside an intracorporeal tissue wall with an elongated shaft having a distal shaft region, on which a means at least partially comprising an elastomer material is provided, which can be reversibly transferred from a position flush with the shaft into a position radially protruding over the shaft, and a needle guide unit provided on the shaft, in which at least one needle, arranged movably along the shaft, is guided. The needle can be reversibly transferred from a retracted position into an advanced distal position, in which the needle penetrates the means located in the position radially protruding over the shaft, wherein by retracting the needle into the retracted position, a suture carried along by the means remains in the means, has been further developed to such extent that the means has limbs that can be radially expanded away from the shaft, which are operatively connected to an element such that the limbs can be reversibly transferred from the position flush with the shaft into the position radially protruding over the shaft.

Just like the surgical suturing apparatus according to U.S. Pat. No. 5,954,732, the surgical suture positioning system according to the invention uses the elastic characteristics of an elastomer material to form a suture loop in the area of a tissue opening to be closed. Unlike the known suturing apparatus, however, the radial expansion of the elastomer element is neither achieved within the scope of material compression nor is the elastomer element subject to an associated material-dependent limitation relative to maximum radial expansion. The radial expansion of the elastomer material is rather predicated on the selection of shape and size of a shaped body having at least two expandable limbs in which refer to embodiments will be made in the following. For the purpose of expanding the limbs, these are laterally deflected either using a suitably positioned dilatable element, in the form of an inflatable body, or by moving the shaped elastomer body relative to a displacement contour, along which the limbs slide. In principle, it is also possible to deflect the limbs of the shaped body with a suitably formed and controllably drivable mechanism.

In the state with the limbs expanded, preferably two hollow needles diametrically are positioned opposite the tissue opening, through each of which one continuous surgical suture is threaded such that the suture respectively protrudes at the needle tip of both needles and is respectively guided back directly along the outside of the needle, to penetrate the vascular wall as well as the elastomer limbs directly flush with the inside of the vascular wall and preferably completely.

Subsequently, both hollow needles are retracted on the proximal side but the suture sections which penetrate through the elastomer limbs, however, remain within the limbs due to the elastic clamping or retaining forces which respectively, act on the respective suture sections. Thus, due to their smooth surfaces, the needles can be retracted from the tissue without any problem and without any effort, on the one hand, and on the other hand, the suture sections are subject to much higher friction to the surrounding elastomer material of the limbs due to their rough surface compared to the needle surface, which is why they are retained. The direct frictional contact between the surgical suture and the elastomer material is achieved in the suture section, which respectively runs along the needle outside the needle surfaces. Ultimately, the elastomer limbs have to be returned from the expanded position into a folded initial position, in which it is possible to remove the means in the proximal direction, without causing additional lesions in the tissue area, through the tissue opening to be closed. With this removal process, a suture loop closing the tissue opening is formed, like in a manner similar to the surgical suturing apparatuses described in the description of the art.

The suture positioning system according to the invention may, in principle, also be designed and used with only a single surgical hollow needle. For that, however, it is required to repeat the penetration process described above at least once at a further point of tissue. On the other hand, surgical interventions are perceivable, where there is demand for only one single suture fixation in the tissue area.

For explanation of specific embodiments, reference is made to the following figures.

### BRIEF DESCRIPTION OF THE DRAWINGS

In the following, the invention is described by way of example without restriction of the general inventive concept on the basis of embodiments referring to the figures, wherein:

FIGS. 1a, b and c show three sides of a suture positioning system designed according to the invention;

FIGS. 2a, b and c show different views of elastomer material designed according to the invention;

FIGS. 3a and b show detailed representations of the distal end region of the needle positioning system with a) folded limbs, as well as b) expanded limbs;

FIGS. 4a-d show sequential image representations for the formation of a suture loop at a tissue opening; and

FIGS. 5a and b show an alternative embodiment for an expansion mechanism of the limbs.

### WAYS TO DESIGN THE INVENTION, COMMERCIAL USABILITY

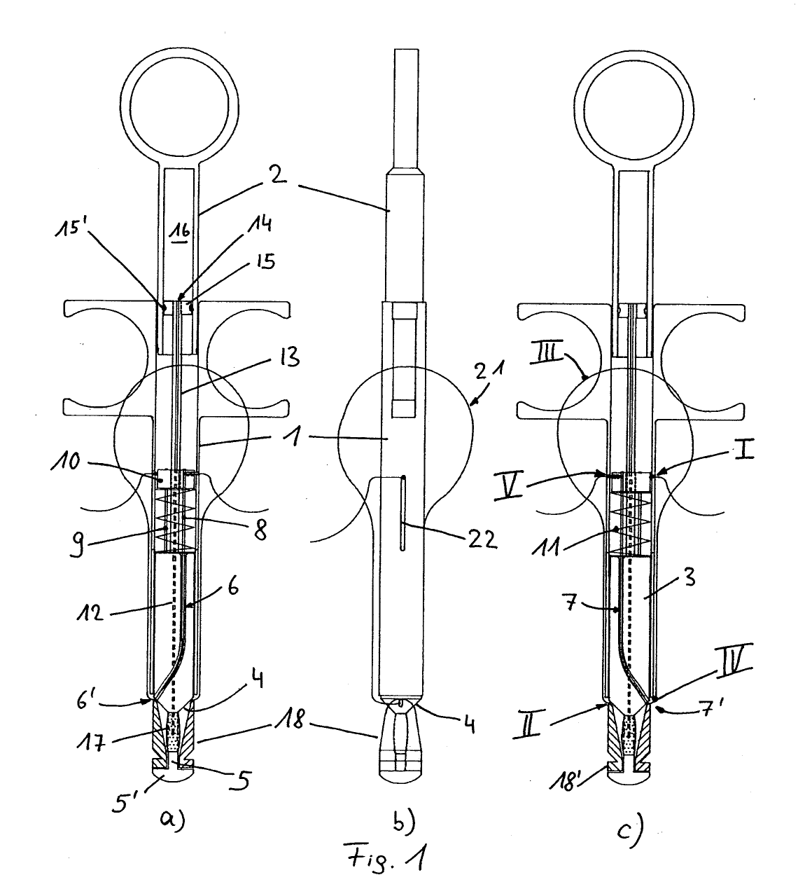

In FIGS. 1a-c, three side views of a surgical suture positioning system designed as a manual device for closing an opening inside an intracorporeal tissue wall is depicted. Suited for handling the needle positioning system are the basic components of a per se known syringe, consisting of a slightly modified syringe body 1 formed in a cylindrical shape as well as a syringe piston 2 guided inside the cylinder-shaped syringe body 1 in a longitudinally movable manner. The syringe piston represents the only actuating element for an operator, which has to be operated in order to perform the entire suturing process. Inside the syringe body 1, which simultaneously serves as the guide tube for the syringe piston 2 as well as for further components arranged inside the syringe piston 1 in a longitudinally movable manner. A shaft 3 is provided in a fixed manner, which protrudes over the syringe body 1 on the distal side and is sealed against the syringe body 1 in a fluid-tight manner. At its distal end region, the shaft 3 provides a conical tapering 4, which is followed by a shaft region 5 with a reduced shaft diameter, which ends in a rounded end cap 5′, which has a cap diameter corresponding to the cross-section of the shaft 3. Preferably, the shaft described above is made from one piece of biocompatible material, that is the shaft sections with the reference numbers 3, 4, 5 and 5′ are made from one piece of uniform material.

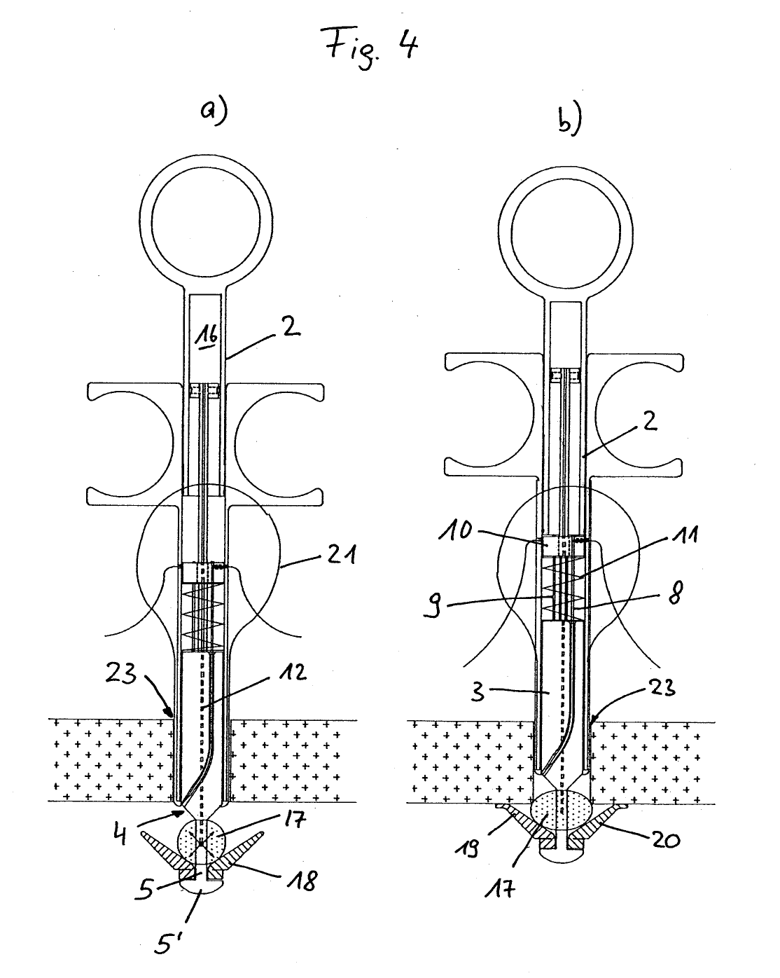

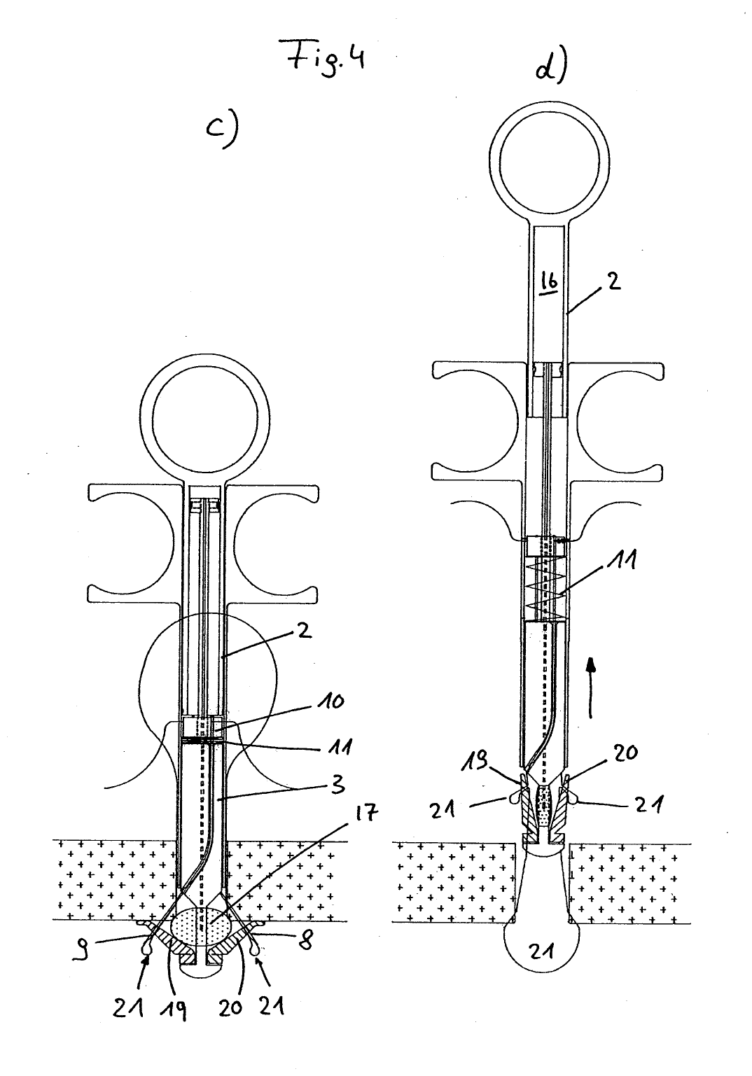

Inside the shaft 3, two needle guide channels 6, 7 are provided (see cross-section front and rear in FIGS. 1a and c), which on the distal side respectively provide a bend with a channel opening 6′ or 7′, respectively, oriented laterally to the longitudinal shaft extension. Inside the respective needle guide channels 6, 7, respectively one needle 8, 9 is mounted movable along the longitudinal shaft axis. The respective proximal needle ends are fixed at a needle guide element 10 which is guided in a longitudinally movable manner inside the syringe body 1. Between the needle guide element 10 and the proximal-side front side of the shaft 3, a spring element 11 is inserted, which is compressed when the needle guide element 10 is deflected towards the shaft 3, which is why simultaneously the hollow needles 8 and 9 mounted inside the needle guide channels 6 and 7 exit from the shaft 3 on the distal side. A more exact functional interconnection of the operatively connected components of the needle positioning system designed according to the invention will be explained in more detail in the following with particular reference to FIG. 4.

Furthermore, a hollow channel 12 runs through the shaft 3 as well as at least partially through the shaft region 5, which is reduced in diameter. The hollow channel 12 is extended rearward to the shaft 3 as a stationary tube 13. The rearward open end 14 of the tube 13 is surrounded by a centering and sealing element 15, which is flush with the inner wall of the syringe piston 2 in a fluid-tight sliding manner. The piston 2 is relative to the syringe body 1 which is mounted in a longitudinally movable manner. Together with the syringe piston 2, the centering and sealing element 15 encloses a fluid-tightly closed volume 16 due to a gasket 15′ provided at the peripheral edge of the centering and sealing element which has a volume filled with a medium, that is preferably air. The distal end of the hollow channel 13 ends inside a dilatable element 17, which is attached at the shaft region 5 which is reduced in cross-section. The dilatable element 17 preferably is designed in the form of an inflatable body surrounding the shaft region 5 in a tubular manner. The inflatable body has an elastically deformable shell wall, which when inflated is toroidal. In case of the embodiment shown in FIGS. 1 a-c, the inflatable body has been emptied and takes a shape which is largely flush with the shaft region 5.

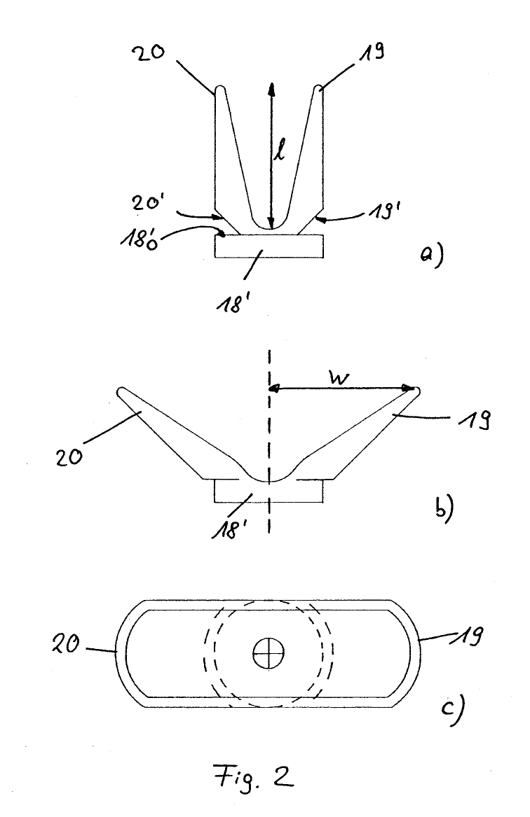

Of particular significance for the suture positioning system designed according to the invention is a shaped body N of an elastomer material or means, which is made from a single piece of an elastomer material comprising the distal shaft region 5 in the manner depicted in FIGS. 1a-c. The material shaped body 18 has a circularly designed base region 18′, which preferably is fixedly connected to the end cap 5′, for example by way of adhesion. In FIGS. 2a-c, detailed representations of the material 18 are respectively shown. The material shaped body 18 preferably is a shaped body made from an elastomer material, having a basic form substantially shaped like a hollow cylinder. The diameter corresponds to the diameter of the shaft 3. Over the base region 18′ two limbs 19 and 20 protrude, which are integrally connected with the base region 18′. The limbs 19 and 20 can be transferred from a position flush with the shaft, as is shown in FIGS. 1a-c as well as 2a, into a radially protruding position over or expanded away from the shaft, as shown, for example, in FIG. 2b. In order to guarantee a defined expansion of the limbs 19 and 20 have a grooved surface 19′ and 20′ facing the base region 18′, which compared to the surface 18′o of the base region 18′ is oriented in an inclined manner. In the expanded state of both limbs 19 and 20, the grooved surfaces 19′ and 20′ respectively rest against the surface 18′o of the base region 18′, such that the swivel range of both limbs 19 and 20 is limited in a defined position.

The lateral swiveling width w illustrated in FIG. 2b can be determined, on the one hand, by the limb length l (see FIG. 2a) as well as, on the other hand, by the orientation of the groove surfaces 19′ and 20;. The elastomer material 18 may particularly advantageously be manufactured from a piece of tubing, which provides the recesses as shown in FIGS. 2a and b. The limbs 19 and 20 thus represent tubing wall sections in the shape of a hollow cylinder and thus have wall sections bent around the central axis. Such a design of the limbs 19, 20 supports a self-driven reset of the expanded limbs into the flush initial position as soon as an external mechanical force for expansion is no longer exerted.

The top view on the expanded limbs 19 and 20 shown in FIG. 2c clarifies the tubing wall-like design of the limbs 19 and 20.

The whole purpose the suture positioning system designed according to the invention is based on the targeted positioning of a surgical suture in the area of a tissue opening for the purpose of closing the opening using the suture. For this reason, reference is made once again to FIGS. 1a-c, on the basis of which the course of the suture through the respective hollow needles 8 and 9 is explained. The suture marked with reference number 21 runs through the needle positions retrievable from FIG. 1c: “I” marks a position inside a longitudinal section 22 in the syringe body 1, through which the suture 21 enters into the proximal end of the hollow needle 8 from the outside, which needle is inserted along the needle guide channel 6. At “II”, the suture 21 exits from the distal opening of the hollow needle 8 and in the case shown is returned towards the proximal end forming a suture sling III. At position IV, the suture 21 enters the distal end of the hollow needle 9, which is provided in the suture guide channel 7. Finally, the suture 21 enters the proximal end of the hollow needle 9 at point V and is likewise led to the outside via a respective slot inside the syringe body.

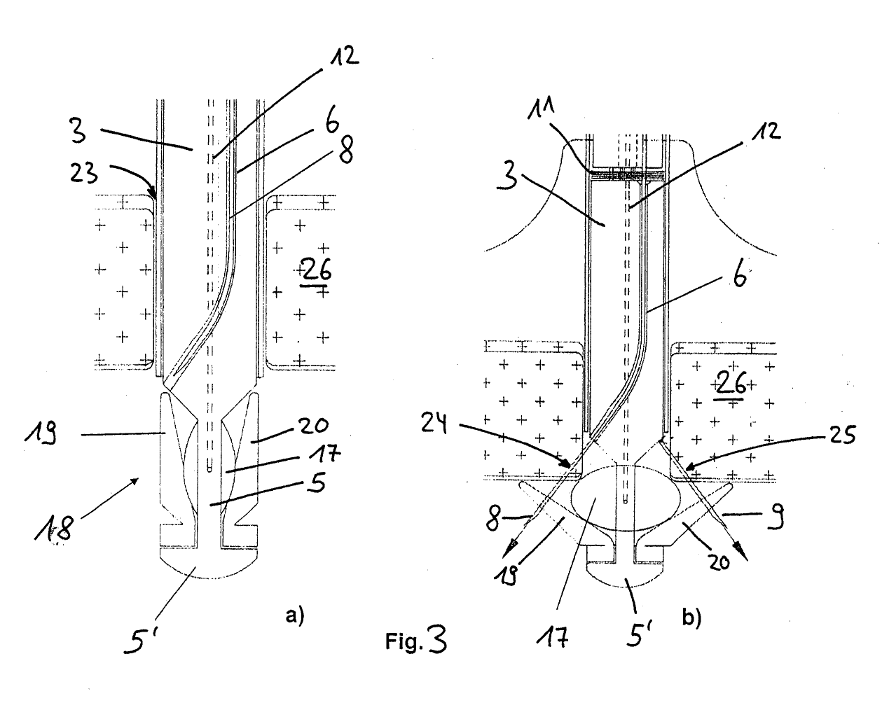

FIG. 3a shows a longitudinal section through the shaft 3, which as one piece passes into the shaft section 5, which is reduced in its cross-section, which finally as one piece passes into the shaft end cap 5′. The means 18 made of elastomer material attached at the distal shaft region takes a position flush with the shaft 3 and in this manner can be positioned through a tissue opening 22. For the sake of explanation only it was to be mentioned that the dilatable element 17 takes a discharged shape, and is thus flush with the shaft region 5. The hollow needle 8 guided inside the needle guide channel 6 also remains in a position completely retracted inside the shaft 3.

In contrast, a state is illustrated in the detail representation according to FIG. 3b, in which the limbs 19 and 20 take an expanded position. For that, the dilatable element 17 is filled with a suitable medium, preferably air, whereby the limbs 19 and 20 are displaced into a position radially expanding away from the shaft. Filling of the dilatable element 17 takes place by feed-in preferably of air along the hollows channel 12 in a manner to be explained in more detail in the following. In an advantageous manner, the dilatable element 17 serves, on the one hand, the centering of the expanded limbs 19 and 20 relative to the tissue opening 22 to be closed. On the other hand, the dilatable element 17 is flush in a fluid-tight manner with the inner wall at the tissue opening 22 and in this manner provides for a fluid-tight seal of the tissue opening 22. Such fluid-tight seal is particularly advantageous for the execution of surgical measures in the abdominal region, in which for the purpose of minimal invasive intervention an intracorporeal excess pressure is generated, which is not affected due to the gas-tight seal explained above.

In the state illustrated in FIG. 3b, needles 8 and 9 are extended along the respective needle guide channels 6 and 7 and in doing so penetrate the laterally expanded limbs 19 and 20. Upon retracting the needles 8 and 9 the suture which is carried remains inside the limbs 19 and 20 which are made of elastomer material due to the mechanism explained above.

In connection with the representation illustrated in FIG. 3b, it is additionally apparent that due to the large lateral expansion width of both limbs 19 and 20, respective needle penetration channels 24 and 25 through the tissue area 26 can be positioned respectively far away from the actual tissue opening, such that the suture carried along through these penetration channels 24 and 25 can hold a sufficient amount of tissue material, with which secure closing of the tissue opening 22 can be guaranteed. In particular the size of the limbs 19 and 20 can be selected to be suitably large, depending on the size of the tissue opening to be closed.

For further description of the mode of operation of the suture positioning system according to the invention as well as for producing an intracorporeal suture loop, and ultimately for the purpose of closing a tissue opening, reference is to be made to the sequential image representation in FIGS. 4a to d.

First of all, at least the regions of the shaft 4, 5 and 5′ protruding over the syringe body 1 on the distal side plus the components provided at this region have to be guided through a tissue opening 22 to be closed. This takes place with the apparatus represented in FIG. 1a, where in particular the means 18 made of elastomer material provides limbs 19 and 20 respectively flush with the shaft 3. When the needle positioning apparatus is situated in the position illustrated in FIG. 4a relative to the tissue opening 22, i.e. at least the shaft regions 4, 5 and 5′ with the elastic means 18 attached thereto as well as the dilatable element 17 are located intracorporeally to the tissue opening 22, the limbs 19 and 20 have to be laterally expanded. For that, the dilatable element 17 is filled with air by the syringe piston 2 being pressed down in a power-actuated manner, whereby the air inside the volume area 16 is pressed through the hollow channel 12 into the dilatable element 17. The dilatable element 17 inflates in the manner shown in FIG. 4a and simultaneously expands the limbs 19, 20.

When the syringe piston 2 gets in contact with the needle guide element 10 by being pushed in further, as is shown in FIG. 4b, then the force to be exerted in order to push in the syringe piston 2 increases noticeably, especially since upon further pushing additionally pushing against the spring force of the spring 11 is necessary. For the operator, this means that by further lowering the syringe piston 2, the needles 8 and 9 are pushed out of the needle guide channels 6 and 7 on the distal side. Before the positioning of the needles takes place, the completely expanded limbs 19 and 20 have to be positioned intracorporeally, centrically to the tissue opening 22. This takes place in a simple manner: the operator lifts the entire suture positioning system in the proximal direction, whereby a noticeable resistance builds up, which prevents further lifting, especially since now the dilated element 17 is pressed against the tissue wall 22 from the inside in a gentle manner. Likewise, the end regions of the limbs 19 and 20 are largely flush with the inner tissue wall, as is shown in FIG. 4b.

Thereafter, the syringe piston 2 is advanced on the distal side against the spring force of the spring 11, whereby the hollow needles 8 and 9, including the suture 21, exit the shaft 3 on the distal side and respectively penetrate a tissue region, which is located at a distance from the tissue opening 22. The penetration process is completed as soon as the syringe piston 2 has reached an end stop, where the spring 11 is completely compressed and the syringe piston 2 lies flush against the proximal-side front side of the shaft 3 along the needle guide element 10 largely directly along the completely compressed spring 11. In this situation, the respective needles 8 and 9 completely penetrate the expanded limbs 19 and 20, as is shown in the sequential image representation according to FIG. 4c. Finally, the needle positioning system has to be retrieved through the tissue opening 22 in the proximal direction, wherein first by retracting the syringe piston 2 the needles 8 and 9 are completely retracted into the shaft 3. This takes place with spring force actuation and independent to a large extent, without an operator having to operate the syringe piston 2 in a force-actuated manner. Once the needles 8 and 9 are completely located inside the shaft 3, wherein the suture 21 is retained inside the respective limbs 19 and 20, the entire suture positioning system is advanced a little on the distal side towards the inside through the tissue opening, such that the expanded limbs 19 and 20 are located at a distance from the inner wall around the tissue opening. In this state not further illustrated in FIG. 4c, the dilated element is vented by retracting the syringe piston 2 completely. In this manner, the needle positioning system takes the shape illustrated in FIG. 4d and can be retracted in the proximal direction from the tissue opening 23. As a result, a surgical suture sling 21 remains on the inside of the tissue opening 23, which subsequently has to be reduced by pulling, whereby the tissue opening is closed.

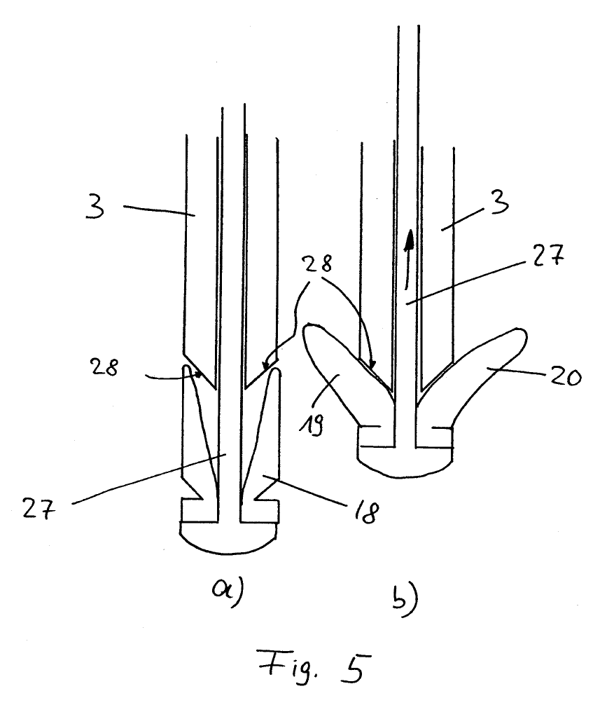

FIGS. 5a and b illustrate an alternative embodiment for an expansion mechanism of the shaped body 18 manufactured from elastomer material. Here, it was assumed that the shaft end cap 5′, as well as the region of the shaft 5 reduced in diameter, are designed as an independent guide element 27, which can be actuated like a stamp, which relative to the shaft 3 is designed and mounted movably in the longitudinal direction, as can be retrieved from the image representation according to FIG. 5a. In the same manner as in the embodiment explained above, the shaped boy 18 made of elastomer material is fixedly attached to the end cap 5′. FIG. 5a shows the state, where the limbs 19 and 20 lie flush against the shaft 3.

Since the distal end of the shaft 3 has a wedge-shaped displacement contour 28, the insides of the limbs 19 and 20 slide along the displacement contour 28, while the guide element 27 is retracted relative to the shaft 3 in the proximal direction. The limbs 19, 20 are automatically expanded due to the displacement contour 27 laterally to the length extension of the shaft.

In order to return the expanded limbs 19 and 20 into the flush position, only the stamp-type guide element 27 has to be advanced. In the alternative embodiment shown in FIG. 5, for reasons of simplified representation, the needle guide system is not shown.

### LIST OF REFERENCE NUMBERS

1 Syringe body
2 Syringe piston
3 Shaft
4 Transition contour
5 Shaft with reduced cross-section
5′ Shaft end cap
6, 7 Needle guide channel
8, 9 Needles, hollow needles
10 Needle guide element
11 Spring
12 Hollow channel
13 Tube
14 Open tube end
15 Centering and sealing element
16 Volume
18 Means, shaped body
18′ Base region
18′o Surface
19, 20 Limbs
19′, 20′ Groove surface
21 Suture
22 Longitudinal section
23 Tissue opening
24 Needle penetration channel
25 Needle penetration channel
26 Tissue area
27 Guide element
28 Displacement contour

1-8. (canceled)

9. A surgical suture positioning system for closing an opening inside an intracorporeal tissue wall, comprising:
an elongated shaft, with a distal region, comprising at least in part an elastomer material, including a body which is reversibly transferred from a position that is flush with the shaft into a position radially protruding beyond the shaft, a needle guide in the shaft in which at least one movable hollow needle is guided so that the needle can be reversibly transferred from a retracted position into an advanced distal position radially protruding beyond the at least one hollow needle wherein by retracting the at least one hollow needle into the retracted position a suture is carried through the body by the at least one hollow needle remaining in the body; and wherein
the body of elastomer material is a hollow cylinder with a circular base from which extends at least two limbs which are respectively expandable laterally relative to a cylindrical axis of the hollow cylinder, the hollow cylinder including a wall bent around the cylinder axis, the limb ends of the body facing away from the circular base region which are oriented in a proximal direction of the shaft, the limbs being expandable away from the shaft and are operatively connected to an element so that the limbs are reversibly transferrable from a position flush with the shaft to a position of radially protruding beyond the shaft; and
the limbs in the position of radially protruding over the shaft are penetrated by the at least one hollow needle, through which the suture passes once and is guided back from the needle tip along an outside of the hollow needle in the proximal direction.

10. The surgical suture positioning system according to claim 9, wherein the needle guide has at least one needle guide channel incorporated into the shaft in which the at least one needle guide channel is bent at a distal end.

11. The surgical suture positioning system according to claim 10, wherein the needle guide has at least two needle guide channels incorporated into the shaft, along which in each channel one hollow needle is movably mounted in a longitudinal direction along the shaft, and tips of the needle are bent at a distal end of the at least two needle guide channels and are mounted diametrically on opposite sides of the shaft at the distal end.

12. The surgical suture positioning system according to claim 9, comprising an element including a dilatable part which can be filled with a medium, located at the distal region and is connected with a channel running inside and along the shaft through which the dilatable part of element can be filled with the medium which can be discharged; and
the dilatable element is supported on the shaft and either rests against the expandable limbs or is fixedly connected therewith.

13. The surgical suture positioning system according to claim 10, comprising an element including a dilatable part which can be filled with a medium, located at the distal region and is connected with a channel running inside and along the shaft through which the dilatable part of element can be filled with the medium which can be discharged; and
the dilatable element is supported on the shaft and either rests against the expandable limbs or is fixedly connected therewith.

14. The surgical suture positioning system according to claim 11,
comprising an element including a dilatable part which can be filled with a medium, located at the distal region and is connected with a channel running inside and along the shaft through which the dilatable part of element can be filled with the medium which can be discharged; and
the dilatable element is supported on the shaft and either rests against the expandable limbs or is fixedly connected therewith.

15. The surgical suture positioning system according to claim 12, wherein on a proximal side of the shaft at least one needle guide element is connected with at least one needle which is movable along the shaft for holding the at least one hollow needle in the retracted position for being activated therefrom;
an actuating element which is movable relative to the shaft in a longitudinal direction which can be engaged with a needle guide and expands it against a spring force along the shaft; and
the actuating element encloses a volume filled with a medium with a volume connected with the channel ending in the dilatable element, through which the medium can be displaced upon expanding the actuating element causing an associated reduction of the medium in the volume into the dilatable element.

16. The surgical suture positioning system according to claim 13, wherein on a proximal side of the shaft at least one needle guide element is connected with at least one needle which is movable along the shaft for holding the at least one hollow needle in the retracted position for being activated therefrom;
an actuating element which is movable relative to the shaft in a longitudinal direction which can be engaged with a needle guide and expands it against a spring force along the shaft; and
the actuating element encloses a volume filled with a medium with a volume connected with the channel ending in the dilatable element, through which the medium can be displaced upon expanding the actuating element causing an associated reduction of the medium in the volume into the dilatable element.

17. The surgical suture positioning system according to claim 14, wherein on a proximal side of the shaft at least one needle guide element is connected with at least one needle which is movable along the shaft for holding the at least one hollow needle in the retracted position for being activated therefrom;
an actuating element which is movable relative to the shaft in a longitudinal direction which can be engaged with a needle guide and expands it against a spring force along the shaft; and
the actuating element encloses a volume filled with a medium with a volume connected with the channel ending in the dilatable element, through which the medium can be displaced upon expanding the actuating element causing an associated reduction of the medium in the volume into the dilatable element.

18. The surgical suture positioning system according to claim 9, wherein the body is attached to a guide crossing the shaft, is movable relative to the shaft and protrudes over the distal shaft region; and
the guide includes a displacement contour formed at the distal shaft region, and which the body can be guided with a relative movement directed in the proximal direction and along which the limbs slide and are expanded radially relative to the shaft.

19. The surgical suture positioning system according to claim 10, wherein the by is attached to a guide crossing the shaft, is movable relative to the shaft and protrudes over the distal shaft region; and
the guide includes a displacement contour formed at the distal shaft region, and which the body can be guided with a relative movement directed in the proximal direction and along which the limbs slide and are expanded radially relative to the shaft.

20. The surgical suture positioning system according to claim 11, wherein the by is attached to a guide crossing the shaft, is movable relative to the shaft and protrudes over the distal shaft region; and
the guide includes a displacement contour formed at the distal shaft region, and which the body can be guided with a relative movement directed in the proximal direction and along which the limbs slide and are expanded radially relative to the shaft.

21. The surgical suture positioning system according to claim 12, wherein the by is attached to a guide crossing the shaft, is movable relative to the shaft and protrudes over the distal shaft region; and
the guide includes a displacement contour formed at the distal shaft region, and which the body can be guided with a relative movement directed in the proximal direction and along which the limbs slide and are expanded radially relative to the shaft.

22. The surgical suture positioning system according to claim 13, wherein the by is attached to a guide crossing the shaft, is movable relative to the shaft and protrudes over the distal shaft region; and
the guide includes a displacement contour formed at the distal shaft region, and which the body can be guided with a relative movement directed in the proximal direction and along which the limbs slide and are expanded radially relative to the shaft.

23. The surgical suture positioning system according to claim 14, wherein the by is attached to a guide crossing the shaft, is movable relative to the shaft and protrudes over the distal shaft region; and
the guide includes a displacement contour formed at the distal shaft region, and which the body can be guided with a relative movement directed in the proximal direction and along which the limbs slide and are expanded radially relative to the shaft.

24. The surgical suture positioning system according to claim 15, wherein the by is attached to a guide crossing the shaft, is movable relative to the shaft and protrudes over the distal shaft region; and
the guide includes a displacement contour formed at the distal shaft region, and which the body can be guided with a relative movement directed in the proximal direction and along which the limbs slide and are expanded radially relative to the shaft.

25. The surgical suture positioning system according to claim 16, wherein the by is attached to a guide crossing the shaft, is movable relative to the shaft and protrudes over the distal shaft region; and
the guide includes a displacement contour formed at the distal shaft region, and which the body can be guided with a relative movement directed in the proximal direction and along which the limbs slide and are expanded radially relative to the shaft.

26. The surgical suture positioning system according to claim 17, wherein the by is attached to a guide crossing the shaft, is movable relative to the shaft and protrudes over the distal shaft region; and
the guide includes a displacement contour formed at the distal shaft region, and which the body can be guided with a relative movement directed in the proximal direction and along which the limbs slide and are expanded radially relative to the shaft.
